(19) United States
(12) Patent Application Publication
(10) Pub. No.: US 2010/0168490 A1
(43) Pub. Date: Jul. 1, 2010

(54) METHOD FOR PREPARATION OF AN ALUMINOSILICATE WITH FERRIERITE STRUCTURE FROM GELS CONTAINING TETRAMETHYLAMMONIUM AND BENZYLMETHYLPYRROLIDINE, AND USES THEREOF

(76) Inventors: Ana Belén Pinar Prieto, Madrid (ES); Joaquín Pérez Pariente, Madrid (ES); Luis Gómez-Hortigüela Sainz, Madrid (ES)

Correspondence Address: IP Patent Docketing;K&L GATES LLP, 599 Lexington Avenue, 33rd Floor, New York, NY, 10022-6030, US

(21) Appl. No.: 12/532,811

(22) PCT Filed: Feb. 28, 2008
(86) PCT No.: PCT/ES08/70035

§ 371 Date: Feb. 12, 2010

(30) Foreign Application Priority Data
Mar. 23, 2007 (ES) P200700782

(51) Int. Cl. C01B 39/04; C01B 39/44; C07C 4/02; C07C 6/00; C07C 2/02

(52) U.S. Cl. 585/533; 423/703; 423/700; 585/649; 585/643

(57) ABSTRACT

The present disclosure comprises a new method for hydrothermal synthesis of ferrierite zeolite, using tetramethylammonium and benzylmethylpyrrodlidine as directional structure agents, and does not require a subsequent process in order to obtain the acid form of the material. This aspect is very important as it avoids subject treatments of calcination and successive interchanges which degrade the structure of the zeolite. The synthetic ferrierite obtained by means disclosed processes has uses in catalytic processes of transformation of hydrocarbons, such as cracking, isomerization, alkylation, and polymerization. Specifically, it can be used in the isomerization of butene to isobutene. To be used in catalytic processes, the zeolite ferrierite must be heated in an oxidant atmosphere in conditions such that the organic material occluded in the interior is eliminated.

### CROSS REFERENCE TO RELATED APPLICATIONS AND PRIORITY CLAIM

This application claims the benefit under 35 U.S.C.§371 to PCT/ES2008/070035 filed Feb. 28, 2008, which claims the benefit of Patent Application P200700782, filed Mar. 23, 2007, in Spain. The entire disclosures of both applications are incorporated by reference herein.

### BACKGROUND

1. Technical Field

This disclosure relates to a new method for preparing zeolite ferrierite, using tetramethylammonium and benzyl-methylpyrrolidinium as structure-directing agents, as well as the use thereof in hydrocarbon conversion reactions. Therefore, this disclosure relates to the preparation of zeolite materials and its application is framed within the chemical industry, more specifically, the petrochemical industry.

2. Background

Ferrierite is a crystalline aluminosilicate that belongs to the group of tectosilicates called zeolites. This zeolite is found in nature as a mineral. The structure of ferrierite is characterised by having a system of channels parallel to the crystal's c-axis direction, delimited by rings formed by 10 tetrahedra, with a size of 4.2×5.4 Å. These channels are connected, by means of rings composed of 8 tetrahedra (size: 3.5×4.8 Å), to small polyhedric cavities called ferrierite type cavities ([5⁸6⁶8²]) the inner diameter whereof is 6×7 Å.

The laboratory synthesis of this zeolite was disclosed, for the first time, by R. M. Barrer and D. T. Marshall (J. Chem. Soc., 484-497 (1964)), who used a method for hydrothermal crystallisation of aqueous gels containing an alkali metal hydroxide, aluminum hydroxide and silica, heated to a temperature between 340° C. and 400° C.

Since then, different methods of obtaining ferrierite using various organic compounds that contain nitrogen, which act as “templates” during the zeolite material crystallisation process, have been published. U.S. Pat. No. 4,251,499 (Feb. 17, 1981) claims the synthesis of ferrierite in the presence of piperidine and alkyl-substituted piperidines, and the sodium cation. U.S. Pat. No. 5,985,238 (Nov. 16, 1999) discloses the hydrothermal synthesis of ferrierite at alkaline pH from gels containing alkali metals and pyridine. All these preparation methods use organic molecules that contain one nitrogen atom and a number of carbon atoms not greater than 6, in the presence of alkaline cations and at alkaline pH.

Ferrierite is used as a catalyst in olefin isomerisation reactions (EP 501,577; EP 523,838); to this end, it must contain acid centres. The generation of these acid centres requires calcination of the material in order to eliminate the organic compounds that are occluded in the interior thereof at the end of the synthesis, the exchange of the alkaline cations present in the material with ammonium ions, and subsequent calcination of the latter to generate the catalytically-active acid centres. Therefore, the methods of preparing ferrierite disclosed thus far require a subsequent treatment to generate acid centres in the material. In this process of calcination and successive exchanges, the structure of zeolite is degraded, and part of the aluminum atoms present therein are removed, which results in a decrease in the material'ₛ catalytic activity.

A new method for preparing ferrierite, using the organic compounds tetramethylammonium and benzyl-methylpyrrolidinium as structure-directing agents, which makes it possible to obtain the material with acid centres without the need for a subsequent treatment that includes calcination steps, is disclosed herein.

### SUMMARY

A first object of the disclosure is a method for preparing zeolite ferrierite by the hydrothermal crystallisation of aqueous gels, characterised by the use of tetramethylammonium and benzyl-methylpyrrolidinium as structure-directing agents.

A particular object of the disclosure is a three-step method for preparing zeolite ferrierite:
(i) preparation of a synthesis mixture,
(ii) thermal treatment of the synthesis mixture and
(iii) separation of the ferrierite crystals,
   such that synthesis mixture (i) comprises, in addition to the compounds tetramethylammonium and benzyl-methylpyrrolidinium, silicon and aluminum compounds, and fluoride ions.

A second object of the disclosure is the Zeolite Ferrierite obtained by the methods disclosed herein.

A third object is the use of the zeolite ferrierite obtained from the disclosed methods in catalytic hydrocarbon conversion processes, such as cracking, isomerisation, alkylation and polymerisation.

### BRIEF DESCRIPTION OF THE FIGURES

FIG. 1 is a diffractogram (CuKα radiation) of the ferrierite obtained following the method described in Example 1. The reflection marked with an * corresponds to traces of quartz.

FIG. 2 is a diffractogram (CuKα radiation) of the ferrierite obtained following the method described in Example 2.

FIG. 3 illustrates the isobutene selectivity of the ferrierites obtained following the methods described in Examples 1 and 2, as compared to the selectivity of a sample of ferrierite obtained following the method described in the patent referred to in Example 3, assayed under the reaction conditions described in Example 3.

### DETAILED DESCRIPTION OF THE PREFERRED EMBODIMENTS

A new method for the hydrothermal synthesis of zeolite ferrierite, using the organic compounds tetramethylammonium and benzyl-methylpyrrolidinium as structure-directing agents, which does not require a subsequent process to obtain the acid form of the material, is disclosed herein. This fact is of great relevance, since it avoids subsequent treatments with calcination and successive exchanges, which degrade the structure of zeolite by removing part of the aluminum atoms present therein and cause a decrease in the materials catalytic activity. Furthermore, this method is characterised in that it is performed from gels that contain fluoride ions, in the absence of inorganic cations.

Methods for preparing zeolite ferrierite by the hydrothermal crystallisation of aqueous gels, characterised by the use of the organic compounds tetramethylammonium and benzyl-methylpyrrolidinium as structure-directing agents are described herein.

Structure-directing agents are defined as those compounds which act as templates during the zeolite material crystallisation process. An essential characteristic of the disclosed methods is the fact that these two structure-directing agents have different sizes, which affects their function during the disclosed method. Thus, tetramethylammonium has a small size and, therefore, fits inside the ferrierite-type cavity, which has a size of about 6 Å, whereas the size of benzyl-methylpyrrolidinium does not allow it to fit inside said cavity, but is sufficiently small to lodge inside the 10-tetrahedra channel. These methods are also characterised in that the hydrothermal synthesis of the zeolite ferrierite crystals does not require the addition of metal, alkali or earth-alkaline, cations, although, optionally, they may be present in the synthesis gel, and in that the synthesis gel contains fluoride ions.

In particular, a method is disclosed wherein tetramethylammonium is in the form of the corresponding hydroxide, or mixtures of tetramethylammonium hydroxide and one or more tetramethylammonium salts, such as chloride, bromide or iodide.

Also described herein is a method wherein benzyl-methylpyrrolidinium is in the form of the corresponding hydroxide, or mixtures of hydroxide and salts such as chloride, bromide or iodide.

The organic additive benzyl-methylpyrrolidinium may be prepared in three steps. The first step comprises alkylation of the secondary amine pyrrolidine with a benzyl halide, whereof benzyl chloride is a non-limiting example, to obtain benzylpyrrolidine. In the second step, the benzyl-methylpyrrolidinium halide, such as, for example, iodide, is prepared by means of a reaction between the benzylpyrrolidine synthesised in the first step and the methyl iodide. In the third step, the benzyl-methylpyrrolidinium iodide may be totally or partially converted into the corresponding hydroxide by means of an ion exchange process, through contact between a benzyl-methylpyrrolidinium iodide solution and an ion exchange resin.

Therefore, one particular object is a method wherein the organic additive benzyl-methylpyrrolidinium is previously prepared by the following three-step process:
alkylation of the secondary amine pyrrolidine with a benzyl halide to produce benzylpyrrolidine;
preparation of the benzyl-methylpyrrolidinium halide by means of the reaction between benzylpyrrolidine and methyl halide; and
ion exchange between the benzyl-methylpyrrolidinium halide and the corresponding hydroxide using an ion exchange resin.

Another particular object is the following three-step method:

(i) Preparation of a synthesis mixture,

(ii) thermal treatment of the synthesis mixture, and

(iii) separation of the ferrierite crystals,

wherein synthesis mixture (i) comprises, in addition to the organic compounds tetramethylammonium and benzyl-methylpyrrolidinium, silicon and aluminum compounds, and fluoride ions.

In one embodiment for carrying out the disclosed method, the fluoride ions come from hydrofluoric acid.

In another embodiment, the silicon compound of synthesis mixture (i) is an alkoxide, such as tetramethoxysilane and tetraethoxysilane, a colloidal silica, or an amorphous or crystalline silica.

In yet another embodiment, the aluminum compound of synthesis mixture (i) is an alkoxide, such as aluminum isopropoxide, or an aluminum salt, such as aluminum nitrate or sulfate, or a hydroxide.

In a further embodiment the synthesis mixture (i) comprises the structure-directing agents tetramethylammonium (TMA) and benzyl-methylpyrrolidinium (BMP), SiO₂, Al₂O₃ and fluoride ions (F), and its chemical composition is between the following ranges expressed in moles:

SiO₂/Al₂O₃: 10-70

(TMA+BMP)/SiO₂=0.30-1.0

TMA/SiO₂=0.005-0.10

BMP/SiO₂=0.295-0.90

H₂O/SiO₂=4-50

F/SiO₂=0.20-1.5

The synthesis mixture may be prepared in the following manner. Once the solution containing the two structure-directing agents is prepared, the precursor of the silicon compound is added thereto, at a temperature of between 5° C. and 80° C., and kept under stirring at the specified temperature for a period of time between 1 minute and 24 hours. Once the silicon compound is added to the solution of the structure-directing agents, the aluminum compound is added and the resulting mixture is kept under stirring at the specified temperatures and for the specified periods of time. Subsequently, the source of fluoride ions, preferably hydrofluoric acid, is added. The resulting mixture is stirred at a temperature of between 5° C. and 80° C., for a period of time between 1 minute and 24 hours.

In another embodiment, the thermal treatment of synthesis mixture (ii) is performed at between 100° C. and 200° C., and, preferably, between 120° C. and 180° C.

In yet another embodiment, the thermal treatment of synthesis mixture (ii) is performed for a period of time between 24 and 720 hours, and, preferably, between 72 and 360 hours.

The separation of the ferrierite crystals described in (iii) may be performed by filtering the solid product obtained following the thermal treatment of the synthesis mixture and, subsequently, washing with de-ionised water and drying.

The zeolite ferrierite obtained by means of the methods described herein is also disclosed.

The synthetic ferrierite obtained by the disclosed methods has applications in catalytic processes, such as, for example, the isomerisation of butene to isobutene, which require the activation of ferrierite. To do so, it is necessary to heat the zeolite ferrierite obtained by the method describes under such conditions that the organic matter occluded inside the crystals during the synthesis process is eliminated. The activation of zeolite is performed by exposing the latter, under high-temperature conditions, to an oxidising atmosphere, which may contain oxygen, such as, for example, air or ozone, at a temperature of between 150° C. and 600° C.

The calcined material obtained by the method described herein has a chemical composition in the anhydrous state expressed by the following formula:

Al₂O₃:mSiO₂

where the value of m is between 10 and 80, preferably between 20 and 60.

The obtained ferrierite is active and selective in catalytic hydrocarbon conversion processes, such as the isomerisation of olefins and, in particular, the isomerisation reaction from butene to isobutene.

Thus, use of the obtained ferrierite in catalytic hydrocarbon conversion processes, such as cracking, isomerisation, alkylation and polymerisation, is also encompassed herein.

In particular, the use of the obtained ferrierite in the isomerisation of olefins is described.

Further described is the use of the obtained ferrierite in the isomerisation of butene to isobutene.

The isomerisation of butene to isobutene may be performed at a temperature that generally ranges between 200° C. and 500° C., at a pressure between 1 atm and 10 atm. The isobutene selectivity depends on the specific reaction conditions, such as temperature, partial pressure of butene and reaction time. The materials obtained have been assayed as butene isomerisation catalysts under severe de-activation conditions, under the following reaction conditions: T=400° C.; P=1 atm; nitrogen/1-butene molar ratio=4; WHSV=25, using 0.1 grams of catalyst. Under these conditions, the materials obtained lead to an isobutene selectivity greater than 85% for a butene conversion of 10%, whereas, for a 30% conversion, the selectivity is greater than 50%. These selectivity results are very high, taking into consideration the severe reaction conditions, and give an idea of the potential that these catalysts have in olefin isomerisation reactions.

Throughout the description and the claims, the word “comprise” and its variants is not intended to exclude other technical characteristics, additives, components or steps. For those skilled in the field, other objects, advantages and characteristics of the invention will become apparent from the disclosure.

The invention will now be described in more detail by way of examples. The following examples are for illustrative purposes only and are not intended, nor should they be interpreted, to limit the scope of the invention.

### EXAMPLES

### Example 1

### Preparation of ferrierite with an Si/Al ratio in the synthesis gel equal to 15.7

This example illustrates the preparation of ferrierite with an Si/Al ratio in the synthesis gel equal to 15.7. Firstly, we describe the synthesis of the benzyl-methylpyrrolidinium cation, which takes place in two steps. In the first step, the tertiary amine benzylpyrrolidine is synthesised, the alkylation whereof, in this case with methyl iodide, leads to the benzyl-methylpyrrolidinium halide.

### Synthesis of the benzyl-methylpyrrolidinium cation

i) Synthesis of the tertiary amine benzylpyrrolidine

The synthesis method for the preparation of the tertiary amine benzylpyrrolidine comprises the alkylation of the secondary amine pyrrolidine with benzyl chloride. In order to prepare 73.0 g of benzylpyrrolidine, 78.2 g of benzyl chloride are first added, drop by drop, to a solution of 65.9 g of pyrrolidine (50% molar excess) in an organic solvent such as ethanol (200 mL). Subsequently, 128.1 g of potassium carbonate are added, in order to favour the alkylation reaction by displacing the equilibrium through the elimination of the hydrochloric acid. The molar composition of the reaction mixture is:
1.5 pyrrolidine:1.0 benzyl chloride:1.5 potassium carbonate

The reaction is performed in a round-bottom flask with a 1-litre capacity, which is kept under reflux, at 90° C. under magnetic stirring, for 48 h. The tertiary amine is filtered in order to eliminate the excess potassium carbonate, washing this solid with ethanol. The ethanol is eliminated by vacuum evaporation (60° C.) in a rotary evaporator. Optionally, the amine resulting from the reaction may be extracted with an organic solvent such as chloroform. In this case, it is necessary to subsequently eliminate the chloroform by vacuum heating. The potential water residues must be eliminated by the addition of a desiccant, such as potassium carbonate, which is subsequently filtered. The product obtained by any of the two methods is purified by vacuum distillation. Benzylpyrrolidine is the fraction that is distilled at a temperature of about 110° C. at an approximate pressure of 17 mmHg. It is obtained in the form of a colourless oil, with a yield of between 65% and 75%.

ii) Synthesis of the salt of the benzyl-methylpyrrolidinium cation

The method for obtaining the salt of the benzyl-methylpyrrolidinium cation that will be used as the structure-directing agent in the synthesis of zeolites consists of two steps. In the first step, benzyl-methylpyrrolidinium iodide is prepared from the previously obtained benzylpyrrolidine. In the synthesis of zeolites described, the benzyl-methylpyrrolidinium cation may be added to the synthesis gel in its hydroxide form; for this reason, in a second step, the benzyl-methylpyrrolidinium iodide is subjected to an ion exchange process wherein it is totally or partially converted into benzyl-methylpyrrolidinium hydroxide.

Alkylation of the previously synthesised tertiary amine benzylpyrrolidine is performed by the addition, under cold conditions, of said amine (73.0 g) to a solution of 92.9 g of methyl iodide (50% molar excess) in 200 mL of ethanol. The reaction is performed in a round-bottom flask, which, during the first few minutes of the reaction, is introduced in an ice bath. The flask is kept capped, at ambient temperature and under magnetic stiffing, for a period of not less than three days. During this time, the flask must be covered with aluminum foil in order to prevent the photodegradation of the quaternary ammonium salt product. Subsequently, the ethanol is eliminated by vacuum evaporation at a temperature of 60° C. with the help of a rotary evaporator. The resulting solid, orange in colour, is benzyl-methylpyrrolidinium iodide. It is purified by exhaustively washing with diethyl ether until the wash waters are colourless. The solid, which is now yellow in colour, is left to dry at ambient temperature, in order to allow for evaporation of the ether. In this manner, 99.4 g of benzyl-methylpyrrolidinium iodide are obtained (approximate yield of 72%).

Finally, the iodide may be totally or partially converted into the corresponding hydroxide by ion exchange. To this end, a 2 M aqueous solution of the benzyl-methylpyrrolidinium iodide obtained (99.4 g) is prepared and placed in a PE/PP bottle, jointly with 328 g of ion exchange resin. The solution is kept under stiffing at ambient temperature for a period of not less than 3 days. Once this time has elapsed, the resin is eliminated by filtration. The resulting liquid, benzyl-methylpyrrolidinium hydroxide, is assayed with hydrochloric acid in order to determine its concentration. In this case, the quantity of product obtained is 50.7 g (80% yield). The synthesis of the zeolite materials described herein is performed with a very small quantity of water; therefore, it is necessary to concentrate the aqueous benzyl methylpyrrolidinium hydroxide solution. To this end, part of the water is vacuum-evaporated in a rotary evaporator, at moderate temperature (not greater than 55° C.), in order to prevent the degradation of the organic compound. Using this method, in this case a 65% solution of benzyl-methylpyrrolidinium hydroxide by weight is obtained.

Subsequently, we describe the synthesis of the zeolite materials following this method, using tetramethylammonium and benzyl-methylpyrrolidinium, the synthesis whereof has been previously described, as structure-directing agents.

iii) Synthesis of the zeolite materials

The zeolite materials with a Si/Al ratio=15.7 in the gel are obtained from synthesis gels with the following molar composition:

0.06 TMAOH: 0.48 BMP hydroxide: 0.48 HF: 0.031 Al₂O₃: 0.969 SiO₂: 4.6H₂O

where TMAOH refers to tetramethylammonium hydroxide.

The reaction is performed in a polypropylene glass with a 250-mL capacity. In the first place, an aqueous solution of the cations used as structure-directing agents is prepared. To this end, 5.28 g of tetramethylammonium hydroxide (25% aqueous solution by weight) are added to a solution that contains 33.36 g of benzyl-methylpyrrolidinium hydroxide (65% aqueous solution by weight). Subsequently, 48.32 g of tetraethoxysilane and 3.05 g of aluminum isopropoxide are added. The mixture is kept under stirring at ambient temperature until complete evaporation of both the alcohol formed in the hydrolysis of the Si and Al sources and the excess water. Subsequently, 4.69 g of hydrofluoric acid (48% aqueous solution by weight) are added drop by drop. The resulting gel, which has a pH of about 10, is manually homogenised with the aid of a spatula or a mortar. Subsequently, the gel is introduced into steel autoclaves with teflon liners (20-mL capacity), which are subjected to the static hydrothermal treatment at 150° C. for selected periods of time. The solid products are filtered, washed with ethanol and water, and allowed to dry at ambient temperature overnight.

The diffractogram of the solid obtained is shown in FIG. 1, where the characteristic diffraction pattern of ferrierite may be observed. The solid's chemical composition is Si_{33.8}Al_{2.2}O₇₂. The dry solid product is subjected to calcination in an air atmosphere, and a 14% weight loss is observed, whereof at least 13.5% corresponds to organic matter and the rest corresponds to water.

### Example 2

### Preparation of Ferrierite with an Si/Al Ratio in the Synthesis Gel Equal to 11

This example illustrates the preparation of ferrierite with an Si/Al ratio in the synthesis gel equal to 11. The synthesis of the benzyl-methylpyrrolidinium cation is performed following the method described in Example 1.

This material is obtained from a synthesis gel with the following molar composition:

0.06 TMAOH:0.48 BMP hydroxide: 0.453 HF: 0.043 Al₂O₃: 0.957 SiO₂: 4.6H₂O

where TMAOH refers to tetramethylammonium hydroxide.

The reaction is performed in a polypropylene glass with a 250-mL capacity. In the first place, an aqueous solution of the cations used as structure-directing agents is prepared. To this end, 5.10 g of tetramethylammonium hydroxide (25% aqueous solution by weight) are added to a solution that contains 33.26 g of benzyl-methylpyrrolidinium hydroxide (65% aqueous solution by weight). Subsequently, 47.37 g of tetraethoxysilane and 4.27 g of aluminum isopropoxide are added. The mixture is kept under stirring at ambient temperature until the complete evaporation of both the alcohol formed in the hydrolysis of the Si and Al sources, and the excess water. Subsequently, 4.41 g of hydrofluoric acid (48% aqueous solution by weight) are added drop by drop. The resulting gel, which has a pH of about 10, is manually homogenised with the aid of a spatula or a mortar. Subsequently, the gel is introduced into steel autoclaves with teflon liners (20-mL capacity), which are subjected to the static hydrothermal treatment at 150° C. for selected periods of time. The solid products are filtered, washed with ethanol and water, and allowed to dry at ambient temperature overnight.

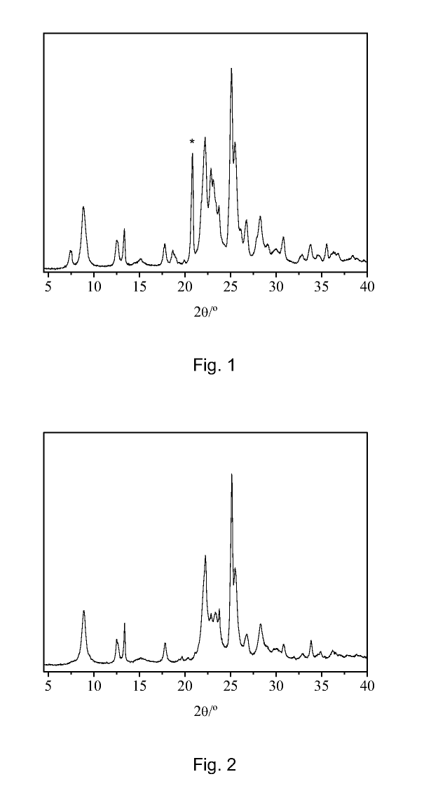

The diffractogram of the solid obtained is shown in FIG. 2, where the characteristic diffraction pattern of ferrierite may be observed. The dry solid product is subjected to calcination in an air atmosphere, and a 15% weight loss is observed, whereof at least 14.5% corresponds to organic matter and the rest corresponds to water.

### Example 3

### Evaluation of the Catalytic Activity of the Ferrierites Prepared Following Examples 1 and 2

This example illustrates the behaviour of the samples obtained following the methods described in Examples 1 and 2 as reaction catalysts in the isomerisation of butene to isobutene. For comparative purposes, a catalytic activity assay of a reference sample of zeolite ferrierite has been included, prepared following the method described in Example 1 of U.S. Pat. No. 4,016,245 (1977), which is directed to the preparation of a zeolite ferrierite from gels that contain sodium ions and the amine pyrrolidine.

In order to use the samples obtained following the methods described herein, it is necessary to activate the zeolite, which requires the elimination of the organic matter incorporated into the zeolite crystals during the synthesis process. To this end, both the samples obtained following the Examples and the reference sample were activated by calcination under oxygen flow (4 litres/hour) enriched with ozone (3%-4%), at a temperature of 200° C., for a period of time between 72 h and 120 h. Subsequently, the samples were heated under an air flow for 4 h at a temperature of 550° C.

In the case of the reference sample, in order to exchange the sodium ions with protons and convert the material into an acid catalyst, after the treatment with an ozone flow and the subsequent calcination, the zeolite was twice exchanged with a 1M ammonium chloride solution and, subsequently, filtered, washed, dried and calcined for 3 h at 350° C. and for another 3 h at 550° C.

The reaction was performed in a fixed-bed reactor, under the following conditions: reaction temperature, 400° C.; pressure, 1 atmosphere; nitrogen/1-butene molar ratio=4; WHSV=25, using 0.1 grams of catalyst.

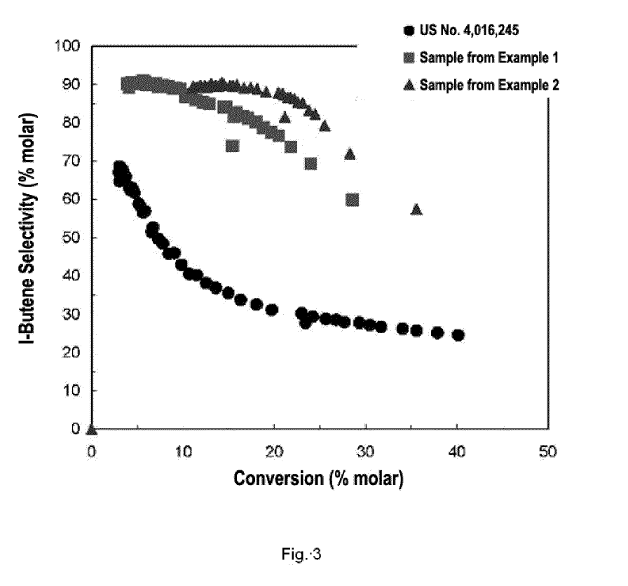

FIG. 3 shows the isobutene selectivity of the three catalysts, and it is clearly observed that the two samples prepared according to the Examples described herein are much more selective toward isobutene than the reference ferrierite sample.

The following table shows the three catalysts, selectivity toward the different reaction products:

| | U.S. Pat. No. | | | | | |
|---|---|---|---|---|---|---|
| Sample | 4,016,245 | | Example 1 | | Example 2 | |
| Conversion (% molar) | 9.84 | 30.48 | 10.27 | 28.58 | 11.01 | 28.29 |
| | Selectivity (% molar) | | | | | |
| Ethylene | 0.00 | 0.41 | 0.00 | 1.09 | 0.00 | 0.62 |
| Propane | 0.00 | 0.35 | 0.00 | 0.81 | 0.00 | 0.38 |
| Propylene | 18.99 | 26.85 | 1.63 | 16.61 | 1.61 | 8.57 |
| Iso-butane | 0.72 | 0.95 | 0.36 | 0.73 | 0.35 | 0.46 |
| N-butane | 1.29 | 1.26 | 0.80 | 4.27 | 0.63 | 2.77 |
| Iso-butene | 42.88 | 27.17 | 86.82 | 59.88 | 88.99 | 71.99 |
| Pentanes | 0.27 | 1.60 | 0.00 | 0.18 | 0.00 | 0.45 |
| Pentenes | 21.73 | 26.88 | 2.10 | 12.94 | 1.95 | 7.52 |
| Hexanes | 5.61 | 11.84 | 0.00 | 2.49 | 0.00 | 3.74 |
| Heptanes | 1.98 | 2.70 | 0.00 | 1.00 | 0.00 | 3.51 |
| C7⁺ | 6.54 | 0.00 | 8.29 | 0.00 | 6.48 | 0.00 |

This table shows that the two samples prepared following the methods described in the Examples have an isobutene selectivity that is at least twice that of the reference ferrierite. In fact, the samples prepared following Examples 1 and 2 show an isobutene selectivity greater than 85% at a 10% conversion, whereas that of the reference zeolite does not exceed 43%. At a 30% conversion, the selectivity of the two samples of Examples 1 and 2 is at least 60%, whereas that of the reference zeolite is less than 30%.

Having sufficiently described the nature of the various example embodiments, it should be stated that the aforementioned processes may have their details modified provided it does not alter the fundamental principle.

The invention is, of course, not limited to the examples described but cover all the variants defined in the claims. The terms “a” and “an” and “the” and similar referents used in the context of the following claims are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g. “such as”) provided herein is intended merely to better illuminate the embodiments and does not pose a limitation on the scope of the embodiments otherwise claimed. No language in the specification should be construed as indicating any non-claimed element essential to the practice of the embodiments.

The use of the term “or” in the claims is used to mean “and/or” unless explicitly indicated to refer to alternatives only or the alternatives are mutually exclusive, although the disclosure supports a definition that refers to only alternatives and“and/or.” Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments are described herein, including the best mode known to the inventors for carrying out the invention. Of course, variations on those preferred embodiments will become apparent to those of ordinary skill in the art upon reading the foregoing description. The inventors expect those of ordinary skill in the art to employ such variations as appropriate, and the inventors intend for the embodiments to be practiced otherwise than specifically described herein. Accordingly, these embodiments include all modifications and equivalents of the subject matter recited in the claims appended hereto as permitted by applicable law. Moreover, any combination of the above-described elements in all possible variations thereof are encompassed by the embodiments unless otherwise indicated herein or otherwise clearly contradicted by context.

Further, it is to be understood that the example embodiments disclosed herein are illustrative. Other modifications that may be employed are within the scope of the embodiments. Thus, by way of example, but not of limitation, alternative configurations of the present embodiments may be utilized in accordance with the teachings herein. Accordingly, the present embodiments are not limited to that precisely as shown and described.

1-15. (canceled)

16. A method for preparing zeolite ferrierite comprising hydrothermal crystallisation of aqueous gels using tetramethylammonium and benzyl-methylpyrrolidinium as structure-directing agents.

17. The method of claim 16, wherein the tetramethylammonium is at least one of tetramethylammonium hydroxide, a tetramethylammonium salt, and mixtures thereof.

18. The method of claim 17, wherein the tetramethylammonium salt is at least one of the chloride salt, the bromide salt, the iodide salt, and mixtures thereof.

19. The method of claim 16, wherein the benzyl-methylpyrrolidinium is at least one of benzyl-methylpyrrolidinium hydroxide, a benzyl-methylpyrrolidinium salt, and mixtures thereof.

20. The method of claim 19, wherein the a benzyl-methylpyrrolidinium salt is at least one of the chloride salt, the bromide salt, the iodide salt, and mixtures thereof.

21. The method of claim 16, wherein the benzyl-methylpyrrolidinium is prepared by a process comprising the steps of:
alkylation of pyrrolidine with a benzyl halide to produce benzylpyrrolidine;
preparation of the benzyl-methylpyrrolidinium halide by reacting the benzylpyrrolidine and methyl halide; and
ion exchange of the benzyl-methylpyrrolidinium halide with the corresponding hydroxide using an ion exchange resin.

22. The method of claim 16, said method comprising the steps of:
(i) preparing a synthesis mixture;
(ii) thermally treating the synthesis mixture; and
(iii) separating ferrierite crystals from the mixture,
wherein the synthesis mixture of step (i) comprises tetramethylammonium (TMA), benzyl-methylpyrrolidinium (BMP), a silicon compound, an aluminum compound, and fluoride ions (F).

23. The method of claim 22, wherein the fluoride ions come from hydrofluoric acid.

24. The method of claim 22, wherein the silicon compound is at least one of an alkoxide, a colloidal silica, an amorphous silica, and a crystalline silica.

25. The method of claim 24, wherein the alkoxide is at least one of tetramethoxysilane and tetraethoxysilane.

26. The method of claim 22, wherein the aluminum compound is at least one of an alkoxide, an aluminum salt, and a hydroxide.

27. The method of claim 26, wherein the alkoxide is aluminum isopropoxide.

29. The method of claim 26, wherein the aluminum salt is at least one of aluminum nitrate and aluminium sulphate.

30. The method of claim 22, wherein the silicon compound comprises SiO₂, the aluminum compound comprises Al₂O₃, and the chemical composition of the synthesis mixture of step (i) is between about the following ranges expressed in moles:
SiO₂/Al₂O₃: 10-70
(TMA+BMP)/SiO₂=0.30-1.0
TMA/SiO₂=0.005-0.10
BMP/SiO₂=0.295-0.90
H₂O/SiO₂=4-50
F/SiO₂=0.20-1.5

31. The method of claim 22, wherein the thermal treatment in step (ii) is performed between about 100° C. and about 200° C.

32. The method of claim 31, wherein the thermal treatment is performed between about 120° C. and about 180° C.

33. The method of claim 22, wherein the thermal treatment in step (ii) is performed for between about 24 and about 720 hours.

34. The method of claim 33, wherein the thermal treatment is performed for between about 72 and about 360 hours.

35. Zeolite ferrierite obtained according to the method of claim 16.

36. Zeolite ferrierite obtained according to the method of claim 34.

37. Use of the zeolite ferrierite of claim 35 in a catalytic hydrocarbon conversion process.

38. The use of zeolite ferrierite according to claim 37, wherein the catalytic hydrocarbon conversion process is at least one cracking, isomerisation, alkylation, and polymerisation.

39. The use of zeolite ferrierite according to claim 37 in olefin isomerisation reactions.

40. The use of zeolite ferrierite according to claim 39 in isomerisation reactions from butene to isobutene.
